(19) United States
(12) Patent Application Publication
(10) Pub. No.: US 2019/0350588 A1
(43) Pub. Date: Nov. 21, 2019

(54) Lung Volume Reduction Apparatus and Methods

(71) Applicant: PneumRx, Inc., Santa Clara, CA (US)

(72) Inventors: Verna Rodriguez, Santa Cruz, CA (US); Kinman Hong, Santa Clara, CA (US)

(73) Assignee: PneumRx, Inc., Santa Clara, CA (US)

(21) Appl. No.: 16/417,183

(22) Filed: May 20, 2019

Related U.S. Application Data
(60) Provisional application No. 62/673,514, filed on May 18, 2018.

(51) Int. Cl. A61B 17/12; A61B 1/267; A61F 2/04

(52) U.S. Cl. CPC A61B 17/12104; A61B 1/2676; A61B 2017/00809; A61F 2/04; A61B 17/12136; A61B 17/1214

(57) ABSTRACT

The invention provides improved medical devices, therapeutic treatment systems, and treatment methods for treatment of the lung. The invention includes methods, systems, and devices for applying a first lung volume reduction action to the functionally impaired lung tissue so as to reduce its volume to less than a pre-treatment volume; and applying a pro-inflammatory stimulus to the functionally impaired lung tissue having reduced volume, that stimulus being sufficient to induce fibrosis in the functionally impaired lung tissue. The pro-inflammatory stimulus may be separate and additional to that of the lung volume reduction action.

### CROSS REFERENCE TO RELATED APPLICATIONS

The present application claims the benefit under 35 USC § 119(e) of U.S. Provisional Appln. No. 62/673,514 filed May 18, 2018, the full disclosure which is incorporated herein by reference in its entirety for all purposes.

### BACKGROUND

The present invention relates to methods, apparatus and systems for the treatment of COPD and particularly emphysema using mechanical and thermal techniques as deployed by medical devices.

Chronic Obstructive Pulmonary Disease (COPD) is a disease that affects the lungs by blocking airflow between the bronchus and alveoli and thus making it difficult to breathe. Emphysema is a form of COPD wherein the tissue that normally holds the lung airways open gets destroyed. Emphysema is characterized by an abnormal and permanent destruction and enlargement of alveoli, thus reducing transfer of oxygen from the lungs to the bloodstream. In emphysema, the inner walls of the alveoli weaken and break, creating one larger air space which reduces the ability of the lungs to take in oxygen and remove carbon dioxide.

Patients with emphysema find it hard to breathe because their airways collapse when they try to exhale, trapping the oxygen depleted air in their lungs and leaving little room for fresh, oxygen-rich air to enter. This “air trapping”, combined with the damaged alveoli and less oxygen transfer to the blood, results in a feeling of breathlessness. As a result, patients with emphysema feel generally exhausted and their exercise capacity is reduced.

One currently available treatment option for patients suffering from emphysema is a surgical procedure called Lung Volume Reduction (LVR) surgery whereby diseased lung is resected and the volume of the lung is reduced. This allows healthier lung tissue to expand into the volume previously occupied by the diseased tissue and allows the movement of the diaphragm to increase its efficiency in transferring gases in an out of healthy tissue. However, high mortality and morbidity may be associated with this invasive procedure.

A further treatment option for patients suffering from emphysema is the use of endobronchial valves. The Zephyr™ device by Ernphasys (Redwood City, Calif.) and the IBV™ device by Spiration (Redmond Wash.) are mechanical one-way valve devices. These devices are placed into the airways supplying air to diseased lung tissue and prevent oxygenated air entering and allowing oxygen depleted air and mucous to pass through the device out of the diseased regions. A limitation of this procedure is that it may not be efficacious in patients with collateral ventilation, that being a condition where other pathways for gas entry into diseased tissue are also present.

### BRIEF SUMMARY

A still further mechanical approach to treating emphysema is by the use of endobronchial nitinol shape memory coils which are delivered by catheter and bronchoscope into the patient's lung. These coils are designed to recoil from a linear form into a ‘coiled’ form once released from a delivery catheter into the lung, causing emphysematous lung tissue to be compressed. Such devices are described in U.S. Patent Applications US 2015/0051709 (U.S. Pat. No. 9,402,633), US 2007/0221230 (U.S. Pat. No. 8,157,837), and US 2009/0076622 (U.S. Pat. No. 8,142,455), which are incorporated herein by reference in their entirety. This presence of the coil restores the elastic properties in adjacent lung tissue and improves the ventilatory mechanical function of the lung. This in turn improves exercise tolerance and symptoms in patients with emphysema and severe lung hyperinflation.

During post hoc safety analysis of the endobronchial coils procedures, chest imaging has identified radiographic opacities in a number of patients. These opacities were thought to indicate localised inflammatory response and investigators originally attributed these opacities to pneumonia related adverse events. However, further investigation has revealed that a high proportion of these opacities were in fact misclassified and did not represent pneumonia related events but rather represented non-infectious coil-associated inflammatory response (“coil associated opacities” or “CAO”).

Upon further investigation, it has now been determined that patients with such coil associated opacities exhibit superior 12-month effectiveness outcomes compared with patients without them. This determination is based upon, inter alia, clinical data illustrated in Table 1 below. This table is an extract from an American Thoracic Society (ATS) 2018 conference, and it is the result of research that was carried out to characterize the radiographic profile of CAO and validate a proposed grading system and then associate the grade with quantitative CT lung volumes and physiologic changes.

TABLE 1

| | | | | Expiratory | |
|---|---|---|---|---|---|
| CAO | % Change in | Change in | Change in | Volume change | 6-minute walk |
| Score | FEV1 | RV (L) | VC (L) | by QCT (mL) | test distance (m) |
| 0 | −2.8 (+/−12.0) | −0.2(+/−0.9) | 0.1 (+/−0.4) | −133.8 (+/−319.1) | 4.9 (+/−61.3) |
| 1 | −0.3 (+/−15.3) | −0.3(+/−0.7) | 0.1 (+/−0.4) | −52.7 (+/−372.4) | 8.3 (+/−64.8) |
| 2 | 3.1 (+/−17.7) | −0.2(+/−0.7) | 0.2 (+/−0.3) | −83.6 (+/−266.0) | −3.5 (+/−61.8) |
| 3 | 10.1 (+/−23.0) | −0.4(+/−0.6) | 0.2 (+/−0.4) | −388.2 (+/−483.3) | 22.0 (+/−76.3) |
| 4 | 17.8 (+/−24.5) | −0.9(+/−0.9) | 0.5 (+/−0.4) | −470.3 (+/−440.8) | 49.0 (+/−78.6) |
| 5 | 22.1 (+/−33.9) | −0.9(+/−1.2) | 0.4 (+/−0.5) | −1289.2 (+/−577.9) | 11.6 (+/−86.8) |

In the context of Table 1, the opacity of CAO is graded on a scale of 0-5, 0 corresponding to no opacity and 5 corresponding to the largest visible level of opacity. In particular, grade 1 corresponds to minimal peri-coil density, 2 corresponds to sub-segmental atelectasis with fibrosis, grade 3 corresponds to atelectasis and fibrosis without cavitation, grade 4 corresponds to segmental atelectasis and fibrosis, and grade 5 corresponds to fibrosis with cavitation.

FEV1, which stands for forced expiratory volume in one second, is a measure of lung function. This represents the volume of air than can forcibly exhaled out by a patient after full inspiration, in one second. Generally, a higher percentage change in FEV1 may be considered a more positive outcome.

RV stands for residual volume and refers to the volume of air remaining in the lungs after a maximal exhalation. Generally, a negative RV change may be an indication of improvement in lung function, thus a more negative RV change, is considered a better outcome.

VC stands for vital capacity and refers to the volume of air breathed out after the deepest inhalation. Generally, the larger the change in VC, the better the outcome.

Expiratory volume change by QCT refers to the change in expiratory volume as measured by quantitative CT.

The 6-minute walk test distance is an indirect measure of lung capacity used in the assessment of respiratory function.

Analysis of the results shown in Table 1 suggests that the presence of CAO does not have a negative effect on respiratory function, as previously thought, but rather, patients with such opacities show greater improvement in respiratory function.

The present inventors have now determined that optimal results, in terms of (i) level of improvement and (ii) robustness and longevity of improvement, can be more reliably obtained by actively causing reduction of diseased lung tissue volume and also separately inducing inflammation in the diseased tissue. For example, inflammation may be induced in a diseased tissue (by one action) following a reduction of lung volume to the reduced tissue (by a separate action). The inflammation may lead to the development of a desired level of fibrosis. Fibrosis may include the formation of excess fibrous connective tissue (or “scar tissue”) in an organ or tissue in response to tissue damage as part of a natural reparative or reactive process. The present inventors have determined that in some embodiments, it may be advantageous to induce fibrosis, for example, to a level such that the CAO score achieves a grade of 3 or 4. By actively inducing this level of response the present inventors have conceived that they can provide optimal FEV1, expiratory volume change improvements and 6-minute walk test results. In some embodiments, inflammation may be induced in a target area by an action that causes limited damage to tissue in the target area. For example, epithelial cells of endobronchial tissue within a target area may be intentionally damaged. The result may be a cascade of responses that may ultimately result fibrosis at or near the area of the damaged tissue. For example, tissue damage may cause the release of inflammatory signals and cytokines that may trigger and inflammatory response and attract immune cells (e.g., macrophages) to the damaged area. These immune cells (and the damaged cells) may release soluble fibrosis mediators such as TGF-β and TGF-α that stimulate fibroblasts. Other soluble mediators of fibrosis that may be released may include CTGF, platelet-derived growth factor (PDGF), and interleukin 4 (IL-4). These mediators may initiate signal transduction pathways such as the AKT/mTOR and SMAD pathways that ultimately lead to the proliferation and activation of fibroblasts. The activated fibroblasts may deposit extracellular matrix into the surrounding connective tissue, resulting in fibrosis that may show up as opacities when imaged.

The present invention now provides a method of reducing the volume of functionally impaired lung tissue in a patient in need of treatment for reduced lung function which embraces this synergistic effect between lung volume reduction and inflammatory response. The method may include applying a first lung volume reduction action to the functionally impaired lung tissue so as to reduce its volume to less than a pre-treatment volume, and then applying a pro-inflammatory stimulus to the functionally impaired lung tissue having reduced volume. Alternatively, the method may include applying a pro-inflammatory stimulus to a functionally-impaired lung tissue first, and then applying a first lung volume reduction action to the functionally impaired lung tissue so as to reduce its volume. Alternatively, the method may include applying the pro-inflammatory stimulus and applying the first lung volume reduction action simultaneously. The pro-inflammatory stimulus may be sufficient to induce fibrosis in the functionally impaired lung tissue. The pro-inflammatory stimulus may be separate and additional to that of the lung volume reduction action.

The lung volume reduction action may be achieved by compacting the diseased tissue. The lung volume reduction action may include the application of one or more of a lung volume reduction coil (LVRC), a one-way bronchial valve, a lung sealant adhesive, active removal of air, vapor ablation, radiofrequency ablation, microwave ablation, electroporation or cryogenic ablation.

The lung volume reduction action may include the active removal of air. The active removal of air may be achieved using an elongate tubular body defining open proximal and distal ends with a passageway suitable for transporting gas extending between the two. The proximal end of the tubular body may be in operational connection with a device for producing a lower pressure than that in the functionally impaired lung tissue, and the distal end may be deployed into the bronchus supplying air to the functionally impaired tissue such that the distal end is located adjacent that functionally impaired tissue. The elongate tubular body may include a catheter. The active removal of air may be achieved by deploying the distal end of the tubular body into the lobar bronchus supplying air to the functionally impaired tissue. The active removal of air may be achieved by applying a reduced pressure to the proximal end of the tubular body such that air in a target area (e.g., a diseased area) located in the functionally impaired tissue adjacent the distal end of the catheter is caused to flow into the catheter, thereby achieving at least partial collapse of alveoli in at least part of the diseased tissue.

The pro-inflammatory stimulus may be applied using a source of heat, cold, sound, mechanical or electrical energy. The stimulus that is applied may be one or more of radiofrequency energy, microwave energy, electroporation, ultrasound energy, vapor and cryogenic cooling. For example, a cryogenic cooling stimulus may be applied by contacting a cryoprobe (or cryogenic ablation probe) or a cryogenic fluid with the functionally impaired tissue. As another example, a vapor stimulus may be applied by contacting vapor with the functionally impaired tissue, and thereby heating it. As another example, a radiofrequency energy stimulus may be applied by applying radiofrequency energy to the functionally impaired tissue using a radiofrequency ablation probe. As another example, the stimulus may be a frictional force that is applied using a tissue-engaging surface of a probe. In some embodiments, the first lung volume reduction action may be applied before the pro-inflammatory stimulus.

The inventive concept also extends to a system for reducing the volume of functionally impaired lung tissue in a patient in need of treatment for reduced lung function. The system may include a first device, which may be a lung volume reduction device adapted for placement adjacent to the functionally impaired lung tissue and operating to reduce the volume of said functionally impaired lung tissue to less than a pre-treatment volume. The system may further include a second device, which may be a device capable of applying a pro-inflammatory stimulus to the functionally impaired lung tissue having reduced volume, that stimulus being sufficient to induce fibrosis in the functionally impaired lung tissue. The first and second devices are deployable together or sequentially, the first followed by the second, to a position adjacent to the functionally impaired lung tissue via the bronchus supplying air to the functionally impaired tissue.

The first and second devices may be deployable together or sequentially. For example, the first device may be deployed followed by the second device. The first and second devices may be deployed to a position adjacent to the functionally impaired lung tissue via the lobar bronchus supplying air to the functionally impaired tissue.

The first and second devices may be deployed through a bronchoscope.

The first device may be a lung volume reduction device selected from a LVRC, a one-way bronchial valve, a lung sealant adhesive applicator, a catheter capable of active removal of air and a vapor ablation catheter.

The second device may be a pro-inflammatory stimulus inducing device selected from a radiofrequency ablation probe, a microwave ablation probe, an electroporation probe, an ultrasound probe, a cryoprobe or an applicator for cryogenic fluid

The first device may include a catheter capable of active removal of air from alveoli of functionally impaired lung tissue whereby that lung tissue is compacted to a volume less than of its pre-treated state.

The first device may include a catheter capable of active removal of air from alveoli of functionally impaired lung tissue whereby that lung tissue is compacted to a volume less than of its pre-treated state.

The catheter may be adapted to guide the second device to the compacted lung tissue, before, during or after operation to induce that compaction.

The system may further include an elongate element capable of being passed down a bronchoscope into a patient's lung via the patient's bronchi. The elongate element may have a first lumen adapted for removal of air from alveoli of target diseased lung tissue and a second lumen adapted for delivery of one or more of a radiofrequency ablation probe, a microwave ablation probe, an electroporation probe, a cryoprobe and an applicator for cryogenic fluid.

The second lumen may be adapted for delivery of one or both of a radiofrequency ablation probe and a cryoablation probe.

In some embodiments, the lung volume reduction action may include deploying an implant device (e.g., an LVRC) from a constrained delivery configuration to an unconstrained deployed configuration in an airway of a lung. The implant device in the unconstrained deployed configuration may be biased to bend the airway of the lung so as to laterally compress a portion of the lung. The implant device may be delivered to the airway of the lung via a first channel of a delivery device and wherein the pro-inflammatory stimulus is applied using a pro-inflammatory stimulus device delivered via a second channel of the delivery device. The implant device may include a coating comprising a sclerosing agent. The pro-inflammatory stimulus may comprise elution of the sclerosing agent from the implant device, wherein the sclerosing agent is configured to damage epithelial tissue of the lung and induce fibrosis.

### BRIEF DESCRIPTION OF THE DRAWINGS

FIG. 1A, FIG. 1B and FIG. 1C illustrate the anatomy of the human respiratory system.

FIG. 1D illustrates a bronchoscope.

FIG. 2A and FIG. 2B illustrate a bronchoscope in combination with a delivery device for a lung volume reduction system according to embodiments of the invention.

FIG. 3A, FIG. 3B and FIG. 3C are schematic illustrations of the lung volume reduction system being used in the lungs.

FIG. 4A illustrates an example of a Lung volume reduction coil (LVRC).

FIG. 4B illustrates a cutaway view of a delivery cartridge system that constrains the LVRC in a deliverable shape.

FIG. 4C illustrates another LVRC that is shaped in a three dimensional shape similar to the seam of a baseball.

FIGS. 4D-4E illustrate other examples of an LVRC.

FIG. 4F illustrates an example of an LVRC showing a covering with perforations adapted and configured to allow the device to be flushed.

FIG. 5 illustrates an example vapor ablation system, with an elongate shaft having a proximal portion and a distal portion.

FIG. 6 illustrates an example of a vapor ablation device delivering vapor into a target area of the lung,

FIG. 7 illustrates an example embodiment of deploying a first device (which may comprise an LVRC) in an airway of a lung and a second device.

FIG. 8 illustrates an example embodiment of a cryogenic fluid applicator being introduced into a patient's lung.

FIG. 9 is a flowchart illustrating a lung volume reduction method according to embodiments of the invention.

FIG. 10 is another flowchart illustrating a lung volume reduction method according to embodiments of the invention.

FIG. 11 is another flowchart illustrating a lung volume reduction method according to embodiments of the invention.

### DETAILED DESCRIPTION

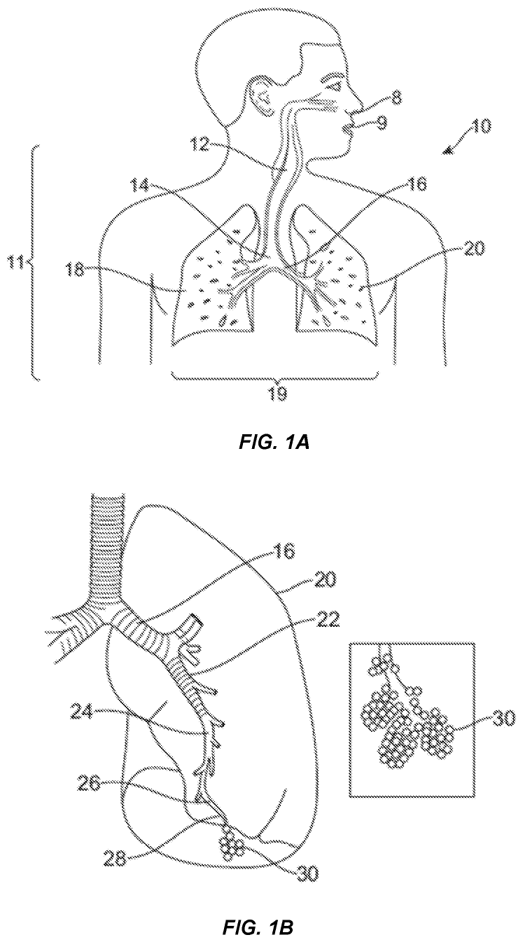

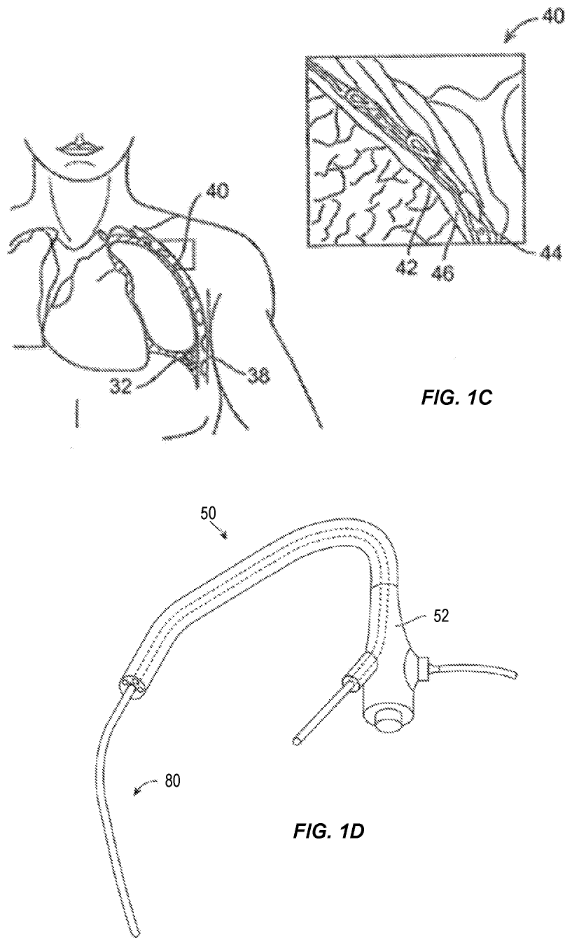

By way of background and to provide context for the invention, FIG. 1A illustrates the respiratory system 10 located primarily within a thoracic cavity 11. This description of anatomy and physiology is provided in order to facilitate an understanding of the invention. Persons of skill in the art, will appreciate that the scope and nature of the invention is not limited by the anatomy discussion provided. Further, it will be appreciated there can be variations in anatomical characteristics of an individual, as a result of a variety of factors, which are not described herein. The respiratory system 10 includes the trachea 12, which brings air from the nose 8 or mouth 9 into the right primary bronchus 14 and the left primary bronchus 16. From the right primary bronchus 14 the air enters the right lung 18; from the left primary bronchus 16 the air enters the left lung 20. The right lung 18 and the left lung 20, together comprise the lungs 19. The left lung 20 is comprised of only two lobes while the right lung 18 is comprised of three lobes, in part to provide space for the heart typically located in the left side of the thoracic cavity 11, also referred to as the chest cavity.

As shown in more detail in FIG. 1B, the primary bronchus, e.g. left primary bronchus 16, that leads into the lung, e.g. left lung 20, branches into secondary bronchus 22, and then further into tertiary bronchus 24, and still further into bronchioles 26, the terminal bronchiole 28 and finally the alveoli 30. As can be seen in FIG. 1C, the pleural cavity 38 is the space between the lungs and the chest wall. The pleural cavity 38 protects the lungs 19 and allows the lungs to move during breathing. The pleura 40 defines the pleural cavity 38 and consists of two layers, the visceral pleurae 42 and the parietal pleurae 44, with a thin layer of pleural fluid therebetween. The space occupied by the pleural fluid is referred to as the pleural space 46. Each of the two pleurae layers 42, 44, are comprised of very porous mesenchymal serous membranes through which small amounts of interstitial fluid transude continually into the pleural space 46. The total amount of fluid in the pleural space 46 is typically slight. Under normal conditions, excess fluid is typically pumped out of the pleural space 46 by the lymphatic vessels.

The lungs 19 are described in literature as an elastic structure that floats within the thoracic cavity 11. The thin layer of pleural fluid that surrounds the lungs 19 lubricates the movement of the lungs within the thoracic cavity 11. Suction of excess fluid from the pleural space 46 into the lymphatic channels maintains a slight suction between the visceral pleural surface of the lung pleura 42 and the parietal pleural surface of the thoracic cavity 44. This slight suction creates a negative pressure that keeps the lungs 19 inflated and floating within the thoracic cavity 11. Without the negative pressure, the lungs 19 collapse like a balloon and expel air through the trachea 12. Thus, the natural process of breathing out is almost entirely passive because of the elastic recoil of the lungs 19 and chest cage structures. As a result of this physiological arrangement, when the pleura 42, 44 is breached, the negative pressure that keeps the lungs 19 in a suspended condition disappears and the lungs 19 collapse from the elastic recoil effect.

When fully expanded, the lungs 19 completely fill the pleural cavity 38 and the parietal pleurae 44 and visceral pleurae 42 come into contact. During the process of expansion and contraction with the inhaling and exhaling of air, the lungs 19 slide back and forth within the pleural cavity 38. The movement within the pleural cavity 38 is facilitated by the thin layer of mucoid fluid that lies in the pleural space 46 between the parietal pleurae 44 and visceral pleurae 42. As discussed above, when the air sacs in the lungs are damaged 32, such as is the case with emphysema, it is hard to breathe. Thus, isolating the damaged air sacs to improve the elastic structure of the lung improves breathing.

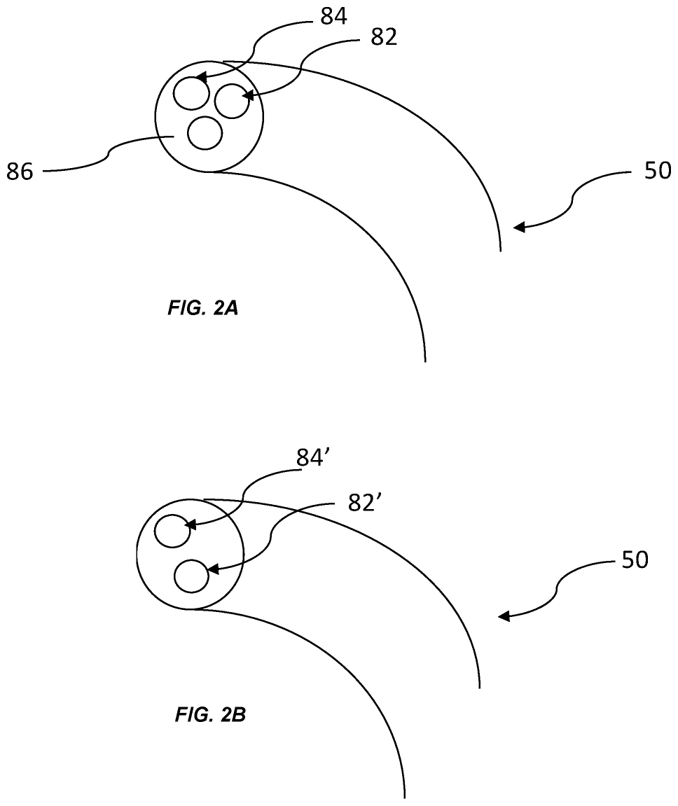

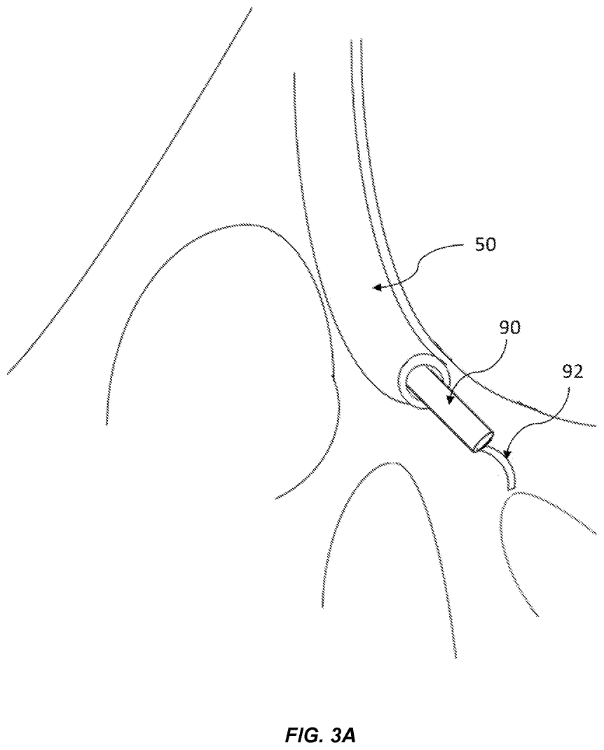

FIG. 1D illustrates the use of a lung volume reduction delivery device 80 for delivering a lung volume reduction system with a bronchoscope 50. As can be seen in FIG. 2A and FIG. 2B, the bronchoscope can be of any suitable length and may include one or more channels suitable for the delivery and deployment of one or more devices. For example, the devices may be one or more of a lung volume reduction coil (LVRC), a one-way bronchial valve, a lung sealant adhesive. FIG. 2A is a schematic illustration of a bronchoscope including three channels 82, 84 and 86. FIG. 2B is a schematic illustration of a bronchoscope including two channels 82′ and 84′. The devices may be deployed contemporaneously, each from a different channel. In some embodiments, the devices may be delivered sequentially, either by the same channel or by different channels.

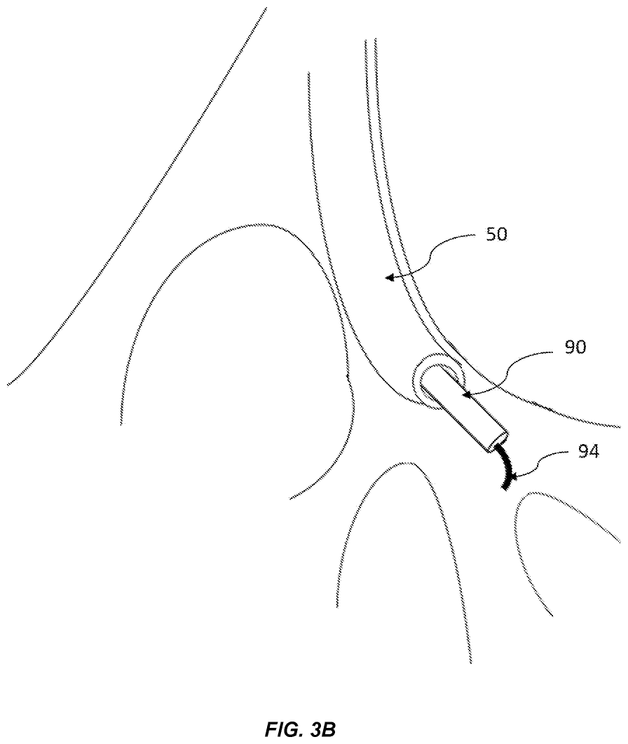

FIG. 3A, FIG. 3D and FIG. 3C illustrate the deployment and use of a system of reducing the volume of functionally impaired lung tissue.

In FIG. 3A, a first device 92 is shown being deployed in an airway. First device 92 may be a lung volume reduction device which is delivered through the bronchoscope 50. In some embodiments, a delivery device 90 is used for the delivery of the first device 92 through the bronchoscope 50. It is to be understood that in some embodiments, the use of catheter 90 is not necessary for the delivery of the first device 92.

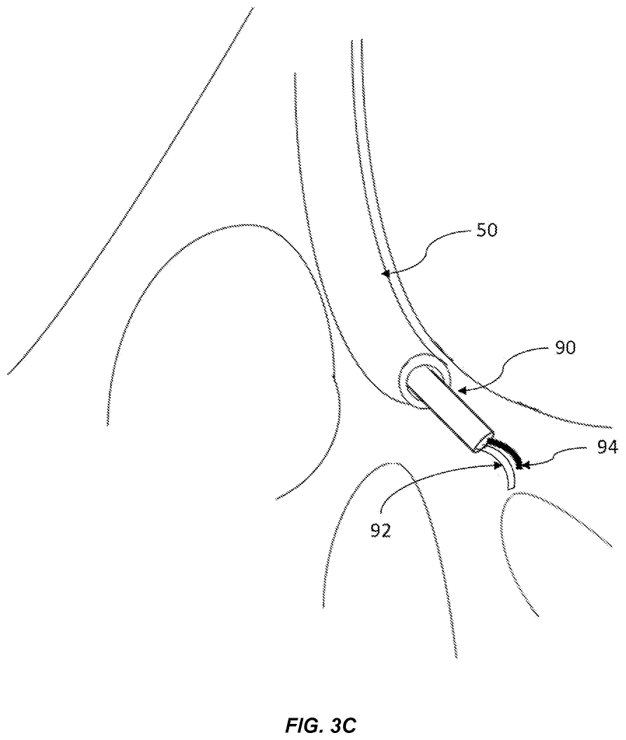

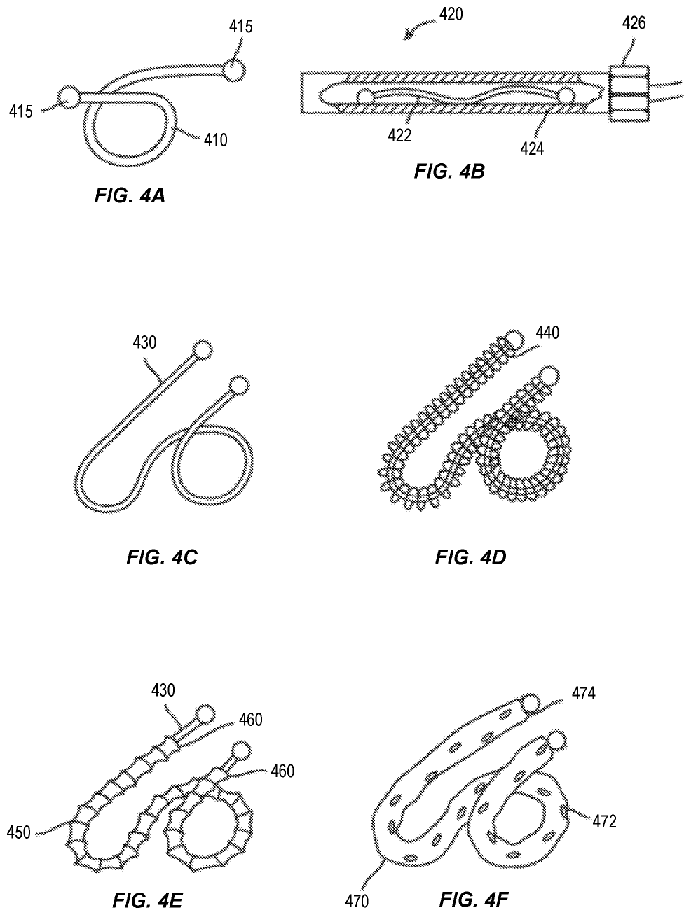

In some embodiments, first device 92 may be a LVRC. In some embodiments, the LVRC may be delivered through the bronchoscope 50 by a delivery device 90. Once released into the lung, the recoils, thus compressing the adjacent lung tissue and achieving lung volume reduction. In some embodiments, delivery device 90 may be used to deliver several coils. FIG. 4A illustrates an example of a LVRC. The NRC may be made from Nitinol metal wire 410. Nickel-Titanium, Titanium, stainless steel or other biocompatible metals with memory shape properties or materials with capabilities to recover after being strained 1% or more may be used to make such an implant. Additionally, plastics, carbon based composites or a combination of these materials would be suitable. The illustrated device in FIG. 4A may be described as being shaped like a French horn and can generally lie in a single plane. The ends are formed into a shape that maximizes surface area shown in the form of balls 415 to minimize scraping or gouging lung tissue. The balls may be made by melting back a portion of the wire, however, they may be additional components that are welded, pressed or glued onto the ends of wire 410.

A Nitinol metallic implant, such as the one illustrated in FIG. 4A, may be configured to be elastic to recover to a desired shape in the body as any other type of spring would or it can be made in a configuration that may be thermally actuated to recover to a desired shape. Nitinol can be cooled to a martensite phase or warmed to an austenite phase. In the austenite phase, the metal recovers to its programmed shape. The temperature at which the metal has fully converted to an austenite phase is known as the Af temperature (austenite final). If the metal is tuned so that the Af temperature is at body temperature or lower than body temperature, the material is considered to be elastic in the body and it will perform as a simple spring. The device can be cooled to induce a martensite phase in the metal that will make the device flexible and very easy to deliver. As the device is allowed to heat, typically due to body heat, the device will naturally recover its shape because the metal is making a transition back to an austenite phase. If the device is strained to fit through a delivery system, it may be strained enough to induce a martensite phase also. This transformation can take place with as little as 0.1% strain. A device that is strain induced into a martensite phase will still recover to its original shape, and convert back to austenite after the constraints are removed. If the device is configured with an Af temperature that is above body temperature, the device may be heated to convert it to austenite and thermally activate its shape recovery inside the body. All of these configurations will work well to actuate the device in the patient's lung tissue. The human body temperature is considered to be 37 degrees C. in the typical human body.

FIG. 4B illustrates a cutaway view of a delivery cartridge system 420 that constrains the LVRC 422 in a deliverable shape. The device 424 may be shipped to the intended user in such a system or it may be used as a tool to more easily load the implant into a desired shape before being installed into the patient, bronchoscope or a catheter delivery device. The cartridge may be sealed or terminated with open, ends or one or more hubs such as the Luer lock hub 426 that is shown. The implant should be constrained to a diameter that is the same or less than 18 mm diameter because anything larger than that will be difficult to advance past the vocal cord opening,

FIG. 4C illustrates another LVRC that is shaped in a three dimensional shape similar to the seam of a baseball. The wire is shaped so that proximal end 430 extends somewhat straight and slightly longer than the other end. This proximal end will be the end closest to the user and the straight section will make recapture easier. If it were bent, it may be driven into the tissue making it hard to access.

FIG. 4D is an illustration of another LVRC. It is similar to that shown in FIG. 39 with the addition of a wire frame 440 surrounding the device. The wire frame may be used, for example, to increase the bearing area that is applied to the lung tissue. By increasing the bearing area, the pressure born by the tissue is reduced along with a reduction in the propensity for the device to grow through lung structures or cause inflammatory issues. Small wires that apply loads in the body tend to migrate so we believe that the device should be configured to possess more than 0.000001 (1⁻⁶ in²) square inches of surface area per linear inch of the length of the LVRC. The frame is one of many ways to provide a larger surface area to bear on the tissue.

FIG. 4E shows yet another example of a LVRC. The LVRC features a covering to increase bearing area. In this example, the main wire 430 is covered by a wire frame and a polymeric covering 450. The covering may be made of any biocompatible plastic, thermoplastic, fluoropolymer, Teflon®, urethane, metal mesh, coating, silicone or other resilient material that will reduce the bearing pressure on the lung tissue. The ends of the covering 460 may remain sealed or open as shown to allow the user to flush antibiotics into and out of the covering.

FIG. 4F illustrates another configuration of the LVRC showing a covering 470 with perforations 472 adapted and configured to allow the device to be flushed. The ends 474 of the covering are sealed to the ends of the device to keep the two components fixed and prevent sliding of one or the other during deployment. The covering may be thermally bonded, glued or shrunk to a tight fit. More information about implantable devices such as LVRCs and their use in reducing lung volume may be found in the following documents, which are incorporated herein by reference in their entirety: U.S. Pat. No. 10,226,257, filed 24 Jun. 2016; U.S. Pat. No. 8,632,605, filed 11 Sep. 2009; U.S. Pat. No. 8,721,734, filed 18 May 2010; U.S. Pat. No. 9,402,633, filed 13 Mar. 2014; U.S. patent application Ser. No. 14/831,007, filed 20 Aug. 2015.

In some embodiments, first device 92 may be a one-way bronchial valve. In some embodiments, the one-way bronchial valve may be delivered through the bronchoscope 50 by a delivery device 90. Once placed in the airway, the bronchial valve allows air to flow through the valve and out of the lung when the patient exhales, but when the patient inhales, the valve closes and blocks air from entering the lung compartment downstream of the valve, thus aiding the lung compartment downstream of the valve to empty itself of air and reducing the overall volume of the lung.

In some embodiments, the first device may be a lung sealant adhesive. A lung sealant delivery device may be delivered through the bronchoscope 50 by a delivery device 90 (e.g., a catheter) to seal pathways into a target area (e.g., a diseased area) and thereby achieve lung volume reduction. In some embodiments, the sealant may be delivered prior or post deployment of a bronchial valve to seal collateral pathways into a target area to increase efficacy of the valves in emptying the tissue of air. In some embodiments, sealant may be delivered in a powder form or a liquid form. In some embodiments, the sealant may be aerosolized.

In some embodiments, the sealant may be a glue composition. The sealant may comprise an adhering moiety that adheres lung tissue, including lung fluids, such as, for example, epithelial lining fluid. An adhering moiety may adhere to lung tissue, for example, sites of non-diseased or normal lung tissue, as well as sites of diseased and/or non-normal lung tissue that may be affected, have been affected, or are likely to be affected by a pulmonary condition. An adhering moiety may bind, attach, or otherwise couple to lung tissue by covalent and/or non-covalent binding. Examples of binding forces that may be useful in the present invention include, but are not limited to, covalent bonds, dipole interactions, electrostatic forces, hydrogen bonds, hydrophobic interactions, ionic, bonds, and/or van der Waals forces. The adhering moiety may adhere to a protease (for example, an elastase) or other molecule and/or macromolecule present in lung tissue. In some embodiments, the adhering moiety may adhere a molecule and/or macromolecule that is bound, attached, coupled, complexed and/or otherwise associated with a cell surface of lung tissue. In some embodiments, the molecule and/or macromolecule may be bound to a cell wall. In some embodiments, the molecule and/or macromolecule may be complexed with a moiety that is itself bound to a cell wall. In some embodiments, the adhering moiety may adhere a molecule and/or macromolecule comprising at least one moiety selected from a protein moiety, a glycoprotein moiety, a lipoprotein moiety, a lipid moiety, a phospholipid moiety, a carbohydrate moiety, a nucleic acid moiety, a modified nucleic acid moiety, and/or a small molecule moiety, including, e.g., a cell surface marker comprising a glycoprotein moiety and/or an ECM component comprising a protein moiety.

In some embodiments, the sealant may be a glue composition that includes a sclerosing agent configured to damage epithelial cells. Introducing the sclerosing agent to lung tissue may cause inflammation and/or fibrosis, e.g., resulting from the damage to the epithelial cells. In some of these embodiments, the first lung volume reduction action and the pro-inflammatory stimulus may comprise the single action of applying the sealant. In other embodiments, an additional pro-inflammatory stimulus may be applied. In these other embodiments, the sclerosing agent may work in conjunction with the pro-inflammatory stimulus to cause inflammation and/or fibrosis. The sclerosing agent may comprise a polycation, Which may be a poly(amino acid). The poly(amino acid) may comprise a plurality of amino acids independently selected from the group consisting of Lys and Arg, and a plurality of amino acids independently selected from the group consisting of Gly, la, Val, Leu, Ile, Met, Pro, Phe, Trp, Asn, Gln, Ser, Thr, Tyr, Cys, and His. In some embodiments, no less than 25 percent of the amino acids may be independently selected from the group consisting of Lys and Arg, and no more than 5 percent of the amino acids may be independently selected from the group consisting of Asp and Glu. The poly(amino acid) may be represented by poly(X-Y), poly(X-Y-Y), or poly(X-Y-Y-Y); X is independently for each occurrence Lys or Arg; and Y is independently for each occurrence Gly, Ala, Val, Leu, Ile, Met, Pro, Phe, Trp. Asn, Gin, Ser, Thr, Tyr, Cys, or His. In some embodiments, the sclerosing agent may be a peroxide (e.g., hydrogen peroxide, a peroxyborate, a peroxyboric acid, a peroxycarbonate, a peroxycarbonic acid, an alkyl hydroperoxide, an aryl hydroperoxide, an aralkyl hydroperoxide, a peroxy acetate, a peroxyacetic acid, sodium perborate, sodium percarbonate, or sodium peracetate). In some embodiments, the sclerosing agent may be a polylysine or a poly(l-lysine). By way of example, the sclerosing agent may comprise one or more of doxycycline, bleomycin, minocycline, doxorubicin, cisplatin+cytarabine, mitoxantrone, Corynebacterium Parvum, streptokinase, and urokinase. In some embodiments, the glue composition may comprise a polymer (e.g., a polyalcohol), a cross-linker (e.g., for causing the polymer and the cross-linker to form a hydrogel), and/or a sclerosing agent. More information about lung sealants, sclerosing agents, and their use in reducing lung volume may be found in U.S. Pat. No. RE416,209, filed 29 Apr. 2015, which is incorporated herein by reference in its entirety.

In some embodiments, first device 92 may be a suction device. The suction device may comprise an elongate device delivered through the bronchoscope 50. The suction device may be delivered to a target area and may actively remove air from the lung compartment downstream of the target area. The target area may be located adjacent lung tissue that is functionally impaired. The suction device may be in the form of an elongate tube configured to be insertable in, and deliverable through, the bronchoscope 50 and suitable for transporting gas such as, by way of example, a suction catheter. The suction device may include a proximal and a distal end and a lumen disposed therebetween. The suction device may be deployed such that its distal end is deployed at or near the target area while its proximal end is connected to a device for producing lower pressure, such as a medical vacuum supply apparatus. By applying a lower pressure at the proximal end of the suction device, air that is present around the distal end of the suction device, at and/or near the target area, flows into the suction device. This airflow out of the target area may cause a partial or complete collapse of the alveoli in the area, thus resulting in reduced lung volume.

In some embodiments, first device 92 may comprise a vapor ablation device for delivering vapor into a target area and thereby achieve lung volume reduction. The device for delivering a vapor may be delivered through the bronchoscope 50. In some embodiments, the vapor may be a condensable vapor generated from a liquid, for example, sterile water or other fluids such as perfluorocarbons, having relatively high liquid-gas phase-change temperatures (i.e. boiling points), preferably temperatures well above body temperature. In some embodiments, the vapor may be at a temperature sufficient to increase the temperature of the surrounding lung parenchyma to cause tissue damage, for example, above at least 40° C. In some embodiments, the vapor delivered by the device may be configured to raise the temperature of the lung tissue in the target area sufficiently high to render at least a portion of the target area essentially non-functional wherein neither blood flow nor air flow occurs within the region. Consequently, at least a portion of the target area may no longer inflate, and lung volume may thereby be reduced. The vapor may rapidly heat the targeted area as the vapor is delivered and may induce tissue collapse, shrinkage, neointima hyperplasia, necrosis and/or fibrosis of the targeted lung region. In some embodiments, the vapor may be delivered to tissue defining an air sac or alveoli within a patient's lung at a temperature above body temperature (for example, about 40° C. to about 80° C., or about 50° C. to about 60° C., at atmospheric pressures) so as to damage the tissue of the air sac or alveoli, the tissue of terminal bronchioles and tissue of collateral passageways. In general the vapor may be applied to the target area through an airway for anywhere from 5 seconds to 10 minutes or longer. In some embodiments, it may be advantageous to deliver the vapor for a relatively short period of time, about 5 seconds to 10 seconds. Short vapor application times may be advantageous in some embodiments, because tissue heating and the resulting damage may be rapid using energetic vapor. In longer procedures, less vapor may be used to cause gradual tissue bioeffects or to treat larger regions or volumes of tissue. Separate procedures may be utilized for separate regions to be treated.

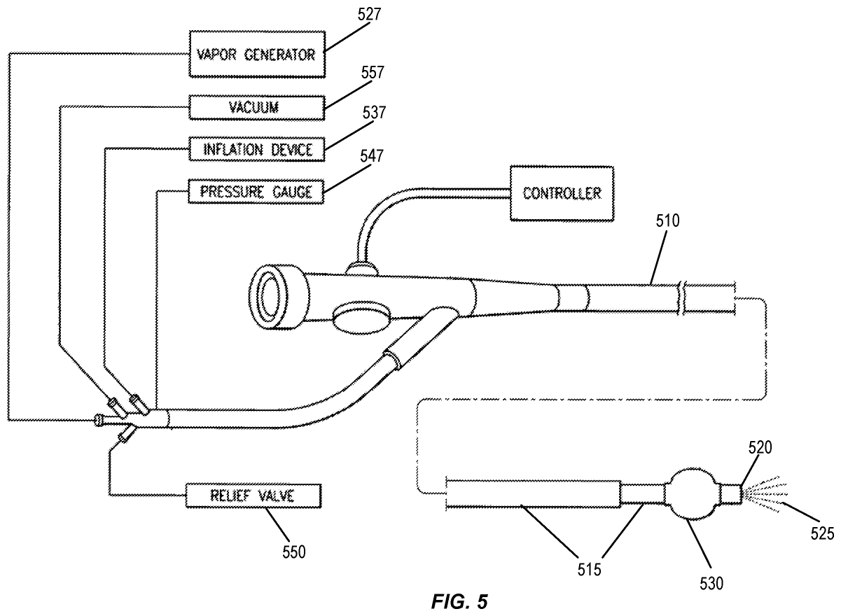

FIG. 5 illustrates an example vapor ablation system, with an elongate shaft having a proximal portion 510 and a distal portion 515. The distal portion 515 may be configured to be delivered within a channel of the bronchoscope 50. The elongated shaft may include at least one discharge port 520 in the distal portion 515 of the elongated shaft configured to discharge vapor 525, which may be conducted through the elongated shaft via a vapor delivering lumen disposed within the elongate shaft in fluid communication with the discharge port 525. A vapor generator 527 may generate the vapor and may be connected to the lumen of the elongate shaft.

In some embodiments, the vapor may be confined to the target area by any suitable means. For example, an occlusion means, such as an inflatable balloon on a balloon catheter, may be used to confine the vapor to the target area and occlude an airway of the lung proximal to the area where the vapor is delivered. Referencing FIG. 5 as an example, the elongated shaft may include within it an inflation lumen which may leads to an inflation port that opens to an interior of the inflatable balloon 530, which may be secured to a location at the distal portion 515 of the shaft. The inflation lumen may be fluidly couple to an inflation device 537 (e.g.; a conventional syringe), which may provide a fluid (e.g., air) via the inflation lumen for inflating the inflatable balloon 530. The inflatable balloon 530 may be configured to prevent vapor flow proximal to the location of the member. Suitable balloon materials may include silicone or latex. The exterior of the working surface of the inflatable balloon 530 may be provided with a knurled or roughened surface to better engage the airway walls and prevent recoil when the condensable vapor is delivered to the target location. In some embodiments, the vapor ablation system may include a pressure sensor on the distal portion 515 of the elongate shaft to detect pressure within the targeted lung region. The pressure sensor may communicates with a pressure gauge 547 on the proximal portion 510 of the elongate shaft. The pressure sensing system may be tied in with a venting system (which may include a relief valve 550) to ensure that preset pressure limits are not exceeded during vapor delivery. Over inflation of the target region could lead to air leaks and tears in the lung pleura.

In some embodiments, to prevent the vapor from entering and damaging adjacent airways and lung regions, the adjacent airways may be filled with a fluid, such as saline. Airways leading to untargeted lung regions may be obstructed to prevent vapor flow therein.

In some embodiments, a vacuum may be applied to the target area after delivery of the condensable vapor to further supplement tissue contraction and collapse caused by introduction of the vapor. Referencing FIG. 5, the elongated shaft may include within it a vacuum lumen (e.g., a hollow lumen) which may lead to an opening at the distal portion 515. The vacuum lumen may be fluidly coupled to a vacuum generator 557. The vacuum generated in the targeted region is about 1 to about 50 mm Hg, preferably about 10 to about 30 mm Hg to effectively collapse the targeted region. The vacuum may also facilitate aspiration of any residual vapor or liquid. More information about vapor ablation devices and their use in lung volume reduction may be found in U.S. Pat. No. 9,050,076, filed 28 Mar. 2011, which is incorporated herein by reference in its entirety.

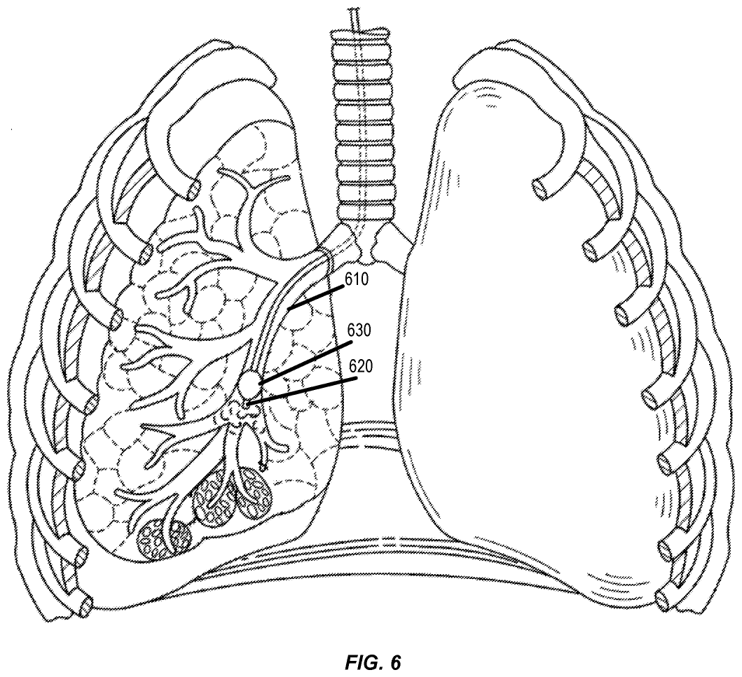

FIG. 6 illustrates an example of a vapor ablation device delivering vapor into a target area of the lung. The vapor ablation device may be advanced through an airway 610 of the lung to the targeted lung region, for example, within a channel of the bronchoscope 50. In some embodiments, the airway 610 may be a bronchial passageway such as segmental bronchi. In some embodiments, the airway 610 may be a sub-segmental bronchi. When a discharge port 620 of the vapor ablation device is at or near the target area, an inflatable balloon 630 may be inflated such that it occludes the airway 610 proximal to the inflatable balloon 630. A desired amount of vapor may then be released from the discharge port 620 toward the target area for one or more desired periods of time.

In some embodiments, first device 92 may comprise an ultrasound probe. In some embodiments, high intensity focused ultrasound (HIFU) energy may be delivered by an ultrasound transducer of the ultrasound probe to damage lung tissue such as the tissue of an air sac or alveoli in the lung. In some embodiments, the ultrasound probe may comprise an elongated shaft and may be advanced into an airway of the lung from within a channel of the bronchoscope 50. A distal portion of the ultrasound probe may be extended out of the channel near the target area. A desired level of HIFU energy may then be emitted by a distal tip of the ultrasound probe. For example, HIFU energy between about 100-10,000 W/cm², may be delivered to one or more focal spots (e.g., circumfererentially around a locus of the airway). The HIFU energy may be delivered in amounts sufficient to cause contraction of lung tissue. Because HIFU can be tightly controlled, the ultrasound energy can be specifically targeted to the epithelium, smooth muscle layer, or collagen layer. Delivery of the HIFU energy can also serve to initiate responses such as neointima hyperplasia, which may further serve to occlude the passageway. The method can include a wave guide to direct the HIFU sound waves to the intended treatment site. Additionally a vacuum may be applied prior the HIFU to draw down the airway or air sacs. Alternatively the vacuum may be applied after delivery of the HIFU energy as in the previously discussed embodiment to further supplement tissue contraction and collapse of the terminal bronchioles, air sacs and collateral passageways caused by introduction of the ultrasound energy.

In some embodiments, first device 92 may comprise a microwave ablation probe. The microwave ablation probe may be directed at the target area to damage tissue in the target area by emission of microwave energy, which may heat the tissue such that it causes damage. In some embodiments, the microwave ablation probe may comprise an elongated shaft and may be advanced into an airway of the lung from within a channel of the bronchoscope 50. A distal portion of the microwave ablation probe may be extended out of the channel near the target area. The probe may comprise a tip that is configured to emit microwave energy. In some embodiments, the tip may comprise an antenna for channelling the microwave energy toward tissue in the target area. The tip may be directed toward tissue in the target area, and microwave energy may be channelled toward the tissue to cause damage to the tissue. In some embodiments, the antenna may be a monopole, dipole, or helical antenna. In monopole and dipole antenna assemblies, microwave energy generally radiates perpendicularly away from the axis of the conductor. Monopole antenna assemblies typically include a single, elongated conductor. A typical dipole antenna assembly includes two elongated conductors that are linearly-aligned and positioned end-to-end relative to one another with an electrical insulator placed therebetween. Helical antenna assemblies include helically-shaped conductor configurations of various dimensions, diameter and length. The main modes of operation of a helical antenna assembly are normal mode (broadside), in which the field radiated by the helix is maximum in a perpendicular plane to the helix axis, and axial mode (end fire), in which maximum radiation is along the helix axis.

In some embodiments, an inner tubular member of the microwave ablation probe may be coaxially disposed around a feedline (which may be any suitable transmission line, e.g., a coaxial cable) and may define a first lumen therebetween. The outer tubular member may be coaxially disposed around the inner tubular member and may define a second lumen therebetween. The microwave ablation probe may include an antenna assembly having a first radiating portion (e.g., a distal radiating section) and a second radiating portion (e.g., a proximal radiating section). The antenna assembly may be operably coupled by the feedline to a transition assembly which may be adapted to transmit the microwave energy to the feedline. The microwave ablation probe may be operably coupled to a microwave generator via a suitable connector assembly. More information about microwave ablation probes may be found in U.S. Pat. No. 9,301,723, filed 15 Mar. 2013, which is incorporated herein by reference in its entirety.

In some embodiments, first device 92 may comprise an electroporation device, a cryoablation probe, or a thermal ablation probe. More information about different types of energy that may be applied to treat lung conditions may be found in PCT Application Publication No. WO2000/062699, filed 21 Apr. 2000, which is incorporated herein by reference in its entirety.

In FIG. 3B a second device 94 is shown being deployed in an airway. Second device 94 may be a device suitable for applying a pro-inflammatory stimulus to a target area in the lung and may be delivered through the bronchoscope 50. The target area may include functionally, impaired tissue. The target area of the second device 94 may be overlapping or adjacent the target area of the first device 92. In some embodiments, the second device 94 may be delivered through a channel of the bronchoscope 50, example via a catheter. It is to be understood that in some embodiments, the use of a catheter may not be necessary for the delivery of the second device 94. In some embodiments, the pro-inflammatory stimulus may be applied after a lung volume reduction action has been performed by any suitable method such as those described above (e.g., after deploying one or more LVRCs). In other embodiments, the pro-inflammatory stimulus may be applied before the lung volume reduction action has been performed. In yet other embodiments, the pro-inflammatory stimulus may be applied simultaneously or approximately simultaneously with the lung volume reduction action.

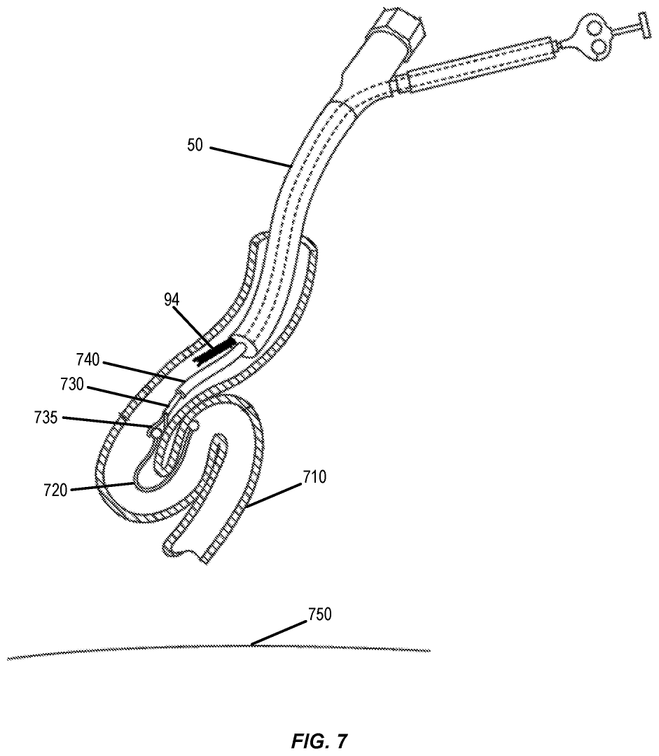

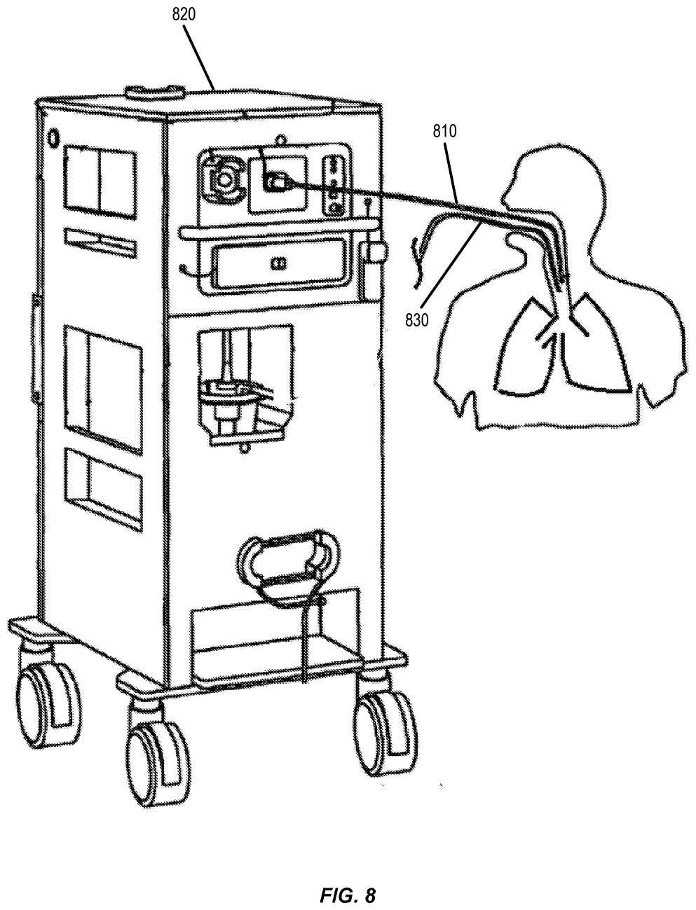

FIG. 7 illustrates an example embodiment of deploying a first device (which may comprise an LVRC) in an airway of a lung and a second device. In some embodiments, the LVRC may be deployed via a first channel of a bronchoscope 50, and the second device may be deployed via a second channel of the bronchoscope. Referencing FIG. 7, a LVRC 720 may be deployed into an airway 710. The LVRC 720 and a pusher 730 may be advanced through a delivery catheter 740 to a location distal to the bronchoscope 50. As illustrated in FIG. 7, the pusher may have grasping jaws 735 that may be locked onto a proximal end of the implant 720, but the implant has recovered to a pre-programmed shape that has also bent the airway 710 into a folded configuration. By folding the airway, the airway structure may be effectively shortened within the lung. Since the airways are well anchored into the lung tissue, the airway provides tension on the surrounding lung tissue which is graphically depicted by showing the pulled (curved inward) floor of the lung 750. The grasper may be used to locate, couple to and retrieve devices that have been released in the patient. It is easy to envision how the implant performs work on the airways and lung tissue without blocking the entire lumen of the airway. This is a benefit in that fluid or air may pass either way through the airway past the implant device. In some embodiments, a second device 94 may be advanced through a second channel of the bronchoscope 50, and extended out of it. The second device 94 may comprise any suitable device described herein. In some embodiments, the second device 94 may comprise a device that emits energy sufficient to damage tissue in the target area. For example, the second device 94 may comprise a vapor ablation probe, a cryoablation probe, a microwave ablation probe, an ultrasound probe, a device configured to mechanically damage tissue, an electroporation device, a thermal ablation probe, or any suitable combination thereof.

In some embodiments, second device 94 may be a radiofrequency ablation probe. The radiofrequency ablation probe may be delivered through the bronchoscope 50 by a delivery, device 90. Once deployed in the target area, the radiofrequency ablation probe is activated to provide thermal energy generated from a radiofrequency alternating current sufficient to cause inflammation of the target area enough to cause fibrosis in the days and weeks after the procedure.

In some embodiments, second device 94 may be a microwave ablation probe. The microwave ablation probe may be delivered through the bronchoscope 50 by a delivery device 90. Once deployed in the target area, the microwave ablation probe is activated using the thermal energy generated from electromagnetic waves in the microwave frequency spectrum sufficient to cause inflammation of the target area enough to cause fibrosis in the days and weeks after the procedure.

In some embodiments, second device 94 may be an electroporation probe. The electroporation probe may be delivered through the bronchoscope 50 by a delivery device 90. Once deployed in the target area, the electroporation probe may apply an electrical field configured to increase the permeability of cells in the affected area sufficient to cause inflammation of the target area enough to cause fibrosis in the days and weeks after the procedure.

In some embodiments, second device 94 may be a cryoprobe (or cryogenic ablation probe). The cryoprobe may be delivered through the bronchoscope 50 by a delivery device 90. Once deployed in the target area, the cryogenic ablation probe may cause ablation by freezing the target area sufficient to cause inflammation of the target area enough to cause fibrosis in the days and weeks after the procedure. In some embodiments, the cryoprobe may be a probe with a tip that is cooled to a low temperature. In some embodiments, the cryoprobe may comprise an elongated shaft and may be advanced into an airway of the lung from within a channel of the bronchoscope 50. A distal portion of the cryoprobe may be extended out of the channel near the target area and caused to contact tissue of the target area.

In some embodiments, second device 94 may be a cryogenic fluid applicator for a cryogenic fluid. The cryogenic fluid applicator may be delivered through the bronchoscope 50 by, a delivery device 90. In some embodiments, the cryoprobe may comprise an elongated shaft and may be advanced into an airway of the lung from within a channel of the bronchoscope 50. A distal portion of the cryoprobe may be extended out of the channel near the target area. Once positioned near the target area, the cryogenic fluid applicator may release cryogenic fluid, such as liquid nitrogen, argon or helium, which may cause freezing upon contact with lung tissue sufficient to cause inflammation of the target area enough to cause fibrosis (e.g., in the days and weeks after the procedure). In some embodiments, the cryogenic fluid applicator may make use of a metered cryospray (e.g., as practiced by the RejuvenAir System of CSA Medical, Inc.). For example, the cryogenic fluid applicator may freeze endobronchial tissue at −196 degrees C. using a pre-determined dose. The dose may be delivered, for example, in a circumferential manner (e.g., via an opening at the distal end of the delivery device, via one or more openings along the circumference of a distal portion of the delivery device). In this example, the dose may be tailored based on the airway size to effect a desired ablation region and depth (e.g., a 10-mm circular ablation with a depth between 0.1 and 0.5 mm). In these embodiments, the epithelium and/or hyperplastic goblet cells may be ablated.

FIG. 8 illustrates an example embodiment of a cryogenic fluid applicator being introduced into a patient's lung. The cryogenic fluid applicator may comprise an elongate shaft 810. In some embodiments, the elongate shaft 810 may be introduced into a patient's lung (e.g., within a catheter) via a conventional therapeutic endoscope, as illustrated in FIG. 8. The endoscope may be of any size, although a smaller diagnostic endoscope may be used for patient comfort. In these embodiments, a first device (e.g., a LVRC, a one-way bronchial valve) may be introduced by a separate delivery device 830. Alternatively, the elongated shaft 810 may be introduced via a second channel of a bronchoscope 50 and a first device (e.g., a LVRC, a one-way bronchial valve) may be introduced via a first channel of the bronchoscope 50. In some embodiments, the elongate shaft 810 may be coupled to a cryogen module 820, which may include a pressurized cryogenic storage tank to store cryogenic fluid (e.g., liquid nitrogen) under pressure. In some embodiments, as the liquid nitrogen travels from the storage tank within the cryogen module 820 to the proximal end of the elongated shaft 810, the liquid may become warmed and may start to boil, resulting in cool gas emerging from the distal end or tip of the elongated shaft 810. The amount of boiling in the elongated shaft 810 may depend on the mass and thermal capacity of the elongated chap 810. In some embodiments, the elongated shaft 810 may be of small diameter (e.g., 7 French) and mass, in which case the amount of boiling may not be great. When the liquid nitrogen undergoes phase change from liquid to gaseous nitrogen, additional pressure may be created throughout the length of elongated shaft 810. When the liquid nitrogen reaches the distal end of the elongate shaft 810, it may be sprayed out of the elongate shaft 810 onto tissue in the target area. While in some embodiments, the liquid nitrogen may be in a substantially gaseous phase, it should be appreciated that in other embodiments, the liquid nitrogen may be in a substantially liquid phase.

In some embodiments, second device 94 may comprise a device for delivering vapor. The device for delivering vapor may be delivered through the bronchoscope 50 by a delivery device 90. Once deployed in the target area, the device may release vapor sufficient to cause inflammation of the target area enough to cause fibrosis in the days and weeks after the procedure. In some embodiments, the vapor may be delivered with microparticulates. Suitable microparticulates may include talc, calcium carbonate, antibiotics such as tetracycline and other penicillin derivates, or other particulate substances which induce fibrosis or cause necrosis of the lung tissue. It will be appreciated that the processes described with reference to FIG. 3A and FIG. 3B may be performed sequentially or contemporaneously. More information about cryoablation devices may be found in the following documents, which are all incorporated herein by reference in their entirety: U.S. Pat. No. 9,301,796, filed 2 Mar. 2012; U.S. Patent Application Publication No. 2016/0242835, filed 20 Feb. 2015; U.S. Patent Application Publication No. 2008/0051776; and PCT Application Publication No. WO 2016/133826, filed 12, Feb. 2016.

In some embodiments, second device 94 may comprise a probe that is configured to mechanically damage tissue (which may be, for example, delivered through the bronchoscope 50 by a delivery device 90). For example, second device 94 may include a tissue-engaging surface of the probe may include an abrasive surface (e.g., it may include an abrasive coating) that may be used to generate a frictional force that may damage epithelial cells along the target area. In this example, the tissue-engaging surface may be rubbed against a portion of the target area in one or more substantially parallel motions to cause shear stress to the epithelium and thereby damage it by a desired amount to cause inflammation and/or fibrosis.

In some embodiments, second device 94 may comprise an ultrasound probe for delivering HIFU energy. Delivery of HIFU energy may be used to trigger fibrosis. As mentioned previously, delivery of HIFU energy can also cause responses such as neointima hyperplasia, which further serves to occlude the airway. In some embodiments, an ultrasound absorptive material, such as a liquid or gel, may be eluted into the airway of the lung. The absorptive material may be heated by the WIT energy in order to thermally damage the surrounding tissue, resulting in contraction of the airway and/or neointima hyperplasia, which may occlude the airway and or damage the air sacs of the lung.

In some embodiments, second device 94 may comprise an electroporation device or a thermal ablation probe (which may be, for example, delivered through the bronchoscope 50 by a delivery device 90). These probes may be used similarly to other probes discussed above, to damage tissue and thereby cause inflammation and fibrosis.

In some embodiments, the first device in the second device may be deployed used in any suitable combination and manner. By way of example, embodiments of the invention may involve the steps of first deploying and using the first device 92, and then deploying and using the second device 94. As another example, embodiments of the invention may involve the steps of first deploying and using the second device 94, and then deploying and using the first device 92. As another example, embodiments of the invention may involve the steps of deploying the first device 92 and the second device 94 together, and alternating use between the first device 92 and the second device 94, As another example, embodiments of the invention may involve the steps of deploying the first device 92 in the second device 94 together, and using the first device 92 before the second device 94 (or alternatively, using the second device 94 before the first device 92).

In some embodiments, the first device 92 may be coated with a pharmaceutical agent that causes fibrosis. In some embodiments, the pharmaceutical agent may comprise one or more sclerosing agents (e.g., one or more of the sclerosing agents disclosed above with respect to the sealant adhesive). In these embodiments, deploying the first device 92 may be a lung volume reduction action and the elution of the pharmaceutical agent may be pro-inflammatory stimulus.

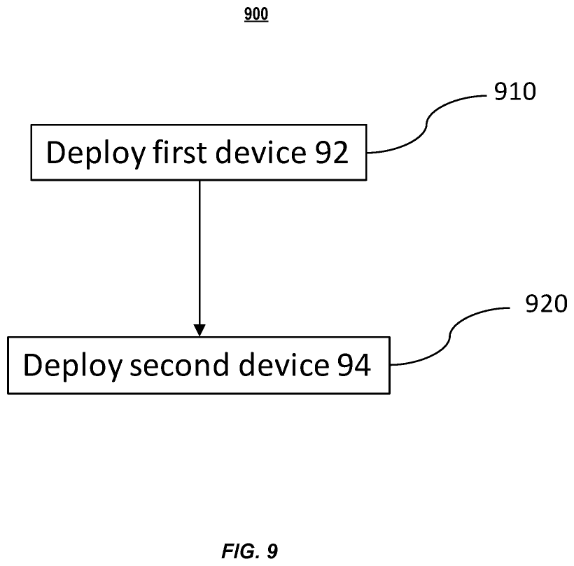

FIG. 9 is a flowchart illustrating an example method 900 for a lung volume reduction method according to embodiments of the invention. The method may begin at step 910, where a first device 92 is deployed, for example, within an airway of a lung at or near a target area. At step 920, a second device 94 is deployed, for example, within the airway of the long at or near the target area. Particular embodiments may repeat one or more steps of the method of FIG. 9, where appropriate. Although this disclosure describes and illustrates particular steps of the method of FIG. 9 as occurring in a particular order, this disclosure contemplates any suitable steps of the method of FIG. 9 occurring in any suitable order. Moreover, although this disclosure describes and illustrates an example method for a lung volume reduction method according to embodiments of the invention, including the particular steps of the method of FIG. 9, this disclosure contemplates any suitable method for a lung volume reduction method according to embodiments of the invention, including any suitable steps, which may include all, some, or none of the steps of the method of FIG. 9, where appropriate. Furthermore, although this disclosure describes and illustrates particular components, devices, or systems carrying out particular steps of the method of FIG. 9, this disclosure contemplates any suitable combination of any suitable components, devices, or systems carrying out any suitable steps of the method of FIG. 9.

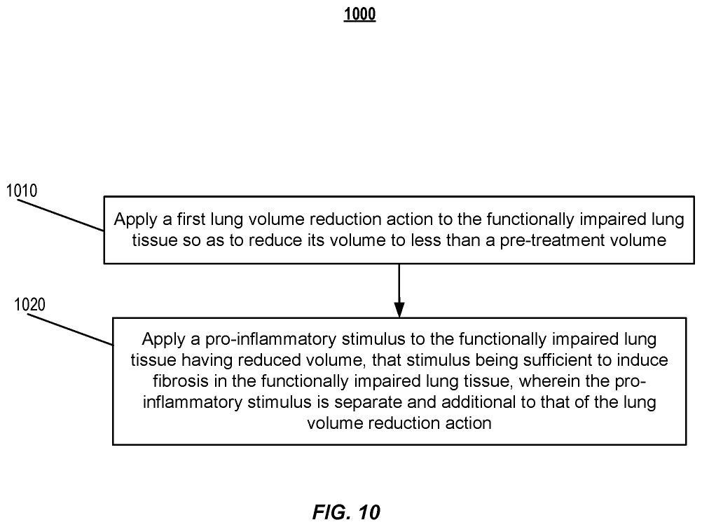

FIG. 10 is a flowchart illustrating an example method 1000 for reducing the volume of functionally impaired lung tissue. The method may begin at step 1010, where a first lung volume reduction action may be applied to the functionally impaired lung tissue so as to reduce its volume less than a pre-treatment volume. At step 1020, a pro-inflammatory stimulus may be applied functionally impaired lung tissue having reduced volume. The stimulus may be sufficient to induce fibrosis in the functionally impaired lung tissue, where in the pro-inflammatory stimulus is separate and additional to that of the lung volume reduction action. Particular embodiments may repeat one or more steps of the method of FIG. 10, where appropriate. Although this disclosure describes and illustrates particular steps of the method of FIG. 10 as occurring in a particular order, this disclosure contemplates any suitable steps of the method of FIG. 10 occurring in any suitable order. Moreover, although this disclosure describes and illustrates an example method for reducing the volume of functionally impaired lung tissue, including the particular steps of the method of FIG. 10, this disclosure contemplates any suitable method for reducing the volume of functionally impaired lung tissue, including any suitable steps, which may include all, some, or none of the steps of the method of FIG. 10, where appropriate. Furthermore, although this disclosure describes and illustrates particular components, devices, or systems carrying out particular steps of the method of FIG. 10, this disclosure contemplates any suitable combination of any suitable components, devices, or systems carrying out any suitable steps of the method of FIG. 10.

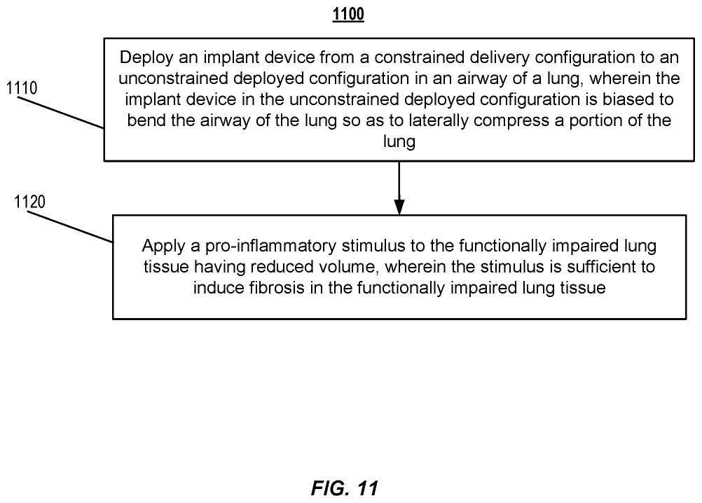

FIG. 11 is a flowchart illustrating an example method 1100 for reducing the volume of functionally impaired lung tissue. The method may begin at step 1110, where an implant device may be deployed from a constrained delivery configuration to an unconstrained deployed configuration in an airway of a lung, wherein the implant device in the unconstrained deployed configuration is biased to bend the airway of the lung so as to laterally compress a portion of the lung. At step 1120, a pro-inflammatory stimulus may be applied to the functionally impaired lung tissue having reduced volume, wherein the stimulus is sufficient to induce fibrosis in the functionally impaired lung tissue. Particular embodiments may repeat one or more steps of the method of FIG. 11, where appropriate. Although this disclosure describes and illustrates particular steps of the method of FIG. 11 as occurring in a particular order, this disclosure contemplates any suitable steps of the method of FIG. 11 occurring in any suitable order. Moreover, although this disclosure describes and illustrates an example method for reducing the volume of functionally impaired lung tissue, including the particular steps of the method of FIG. 11, this disclosure contemplates any suitable method for reducing the volume of functionally impaired lung tissue, including any suitable steps, which may include all, some, or none of the steps of the method of FIG. 11, where appropriate. Furthermore, although this disclosure describes and illustrates particular components, devices, or systems carrying out particular steps of the method of FIG. 11, this disclosure contemplates any suitable combination of any suitable components, devices, or systems carrying out any suitable steps of the method of FIG. 11.

### Example 1: LVRC and RF Application of Thermal Energy

In a first example of the first aspect of the invention, the method deploys a first device, which may be a LVRC, using delivery device 90. As with FIG. 3A, once released into the lung, the LVRC may recoil, compressing the adjacent lung tissue and achieving lung volume reduction. Subsequently, a radiofrequency ablation probe is delivered to the compressed lung tissue, as discussed with reference to FIG. 3B. Once deployed in the target area, the radiofrequency ablation probe creates thermal energy generated from a radiofrequency alternating current sufficient to cause inflammation of the target area enough to cause fibrosis in the days and weeks after the procedure.

### Example 2: LVRC and Microwave Probe Application of Thermal Energy

In a second example of the first aspect of the invention, the method deploys as first device a LVRC using delivery device 90. As with FIG. 3A, once released into the lung endobronchial coil recoils compressing the adjacent lung tissue and achieving lung volume reduction. Subsequently, a microwave ablation probe is delivered to the compressed lung tissue as discussed with reference to FIG. 3B. Once deployed in the target area, the microwave ablation probe creates thermal energy generated from electromagnetic waves in the microwave frequency spectrum sufficient to cause inflammation of the target area enough to cause fibrosis in the days and weeks after the procedure.

### Example 3: LVRC and Electroporation Probe Application of Damage to Tissue

In a third example of the first aspect of the invention, the method deploys a LVRC as in examples 1 and 2. Subsequently, an electroporation probe is delivered as discussed with reference to FIG. 3B, Once deployed in the target area, the electroporation probe applies an electrical field which increases the permeability of cells in the affected area sufficient to cause inflammation of the target area enough to cause fibrosis in the days and weeks after the procedure.

### Example 4: LVRC and Cryogenic Ablation Probe Application of Freezing

In a fourth example of the first aspect of the invention, the method deploys a LVRC as in examples 1 to 3. Subsequently, a cryoprobe is delivered as discussed with reference to FIG. 3B. Once deployed in the target area, the cryogenic ablation probe freezes the target area sufficient to cause inflammation of the target area enough to cause fibrosis in the days and weeks after the procedure.

### Example 5: LVRC and Cryoprobe Application of Freezing to Coil

In a fifth example the method of Example 4 is followed but the cryogenic ablation probe is put in contact with the deployed coil causing the temperature of the coil to drop rapidly, thus changing its elastic properties and causing it to recoil further. This increases the lung volume reduction effect. Additionally, as the coil is metallic and thus an efficient heat conductor, all lung tissue in contact with the coil is also exposed to the low temperature and causing the desired inflammation.

### Example 6: LVRC and Application of Cryogenic Fluid Freezing

In a sixth example of the first aspect of the invention, the method deploys a LVRC as described in Examples 1 to 5. Subsequently, an applicator for a cryogenic fluid is deployed, as described with reference to FIG. 3B. Once deployed in the target area, the cryogenic fluid applicator releases cryogenic fluid, such as liquid nitrogen, liquid argon or liquid helium, which causes rapid freezing upon contact with lung tissue process freezes the target area sufficient to cause inflammation of the target area enough to cause fibrosis in the days and weeks after the procedure.

### Example 7: LVRC and Application of Thermal Energy by Vapor

In a seventh example of the first aspect of the invention, the method deploys a LVRC as described in Examples 1 to 6. Subsequently a device for delivering vapor is deployed, as described with reference to FIG. 3B, Once deployed in the target area, the device releases vapor which generates heat in the target lung tissue sufficient to cause inflammation of the target area enough to cause fibrosis in the days and weeks after the procedure.

### Example 8: Bronchial Valve and Application of Thermal Energy by Vapor

In an eighth example of the first aspect of the invention, the method deploys a one-way bronchial valve delivered as discussed with reference to FIG. 3A. Once placed in the airway, the bronchial valve allows air to flow through the valve and out of the lung when the patient exhales, but when the patient inhales, the valve closes and blocks air from entering the lung compartment downstream of the valve, thus aiding the lung compartment downstream of the valve to empty itself of air and reducing the overall volume of the lung. Subsequently, a vapor application device is delivered, as discussed with reference to FIG. 3B. Once deployed in the target area, the device releases vapor which generates heat in the target lung tissue sufficient to cause inflammation of the target area enough to cause fibrosis in the days and weeks after the procedure.

### Example 9: Bronchial Valve and Application of Thermal Energy by Microwave

In a ninth example of the first aspect of the invention, the method deploys a one-way bronchial valve as described in Example 8. Subsequently, a microwave ablation probe may be delivered as discussed with reference to FIG. 3B. Once deployed in the target area, the microwave ablation probe creates thermal energy generated from electromagnetic waves in the microwave frequency spectrum sufficient to cause inflammation of the target area enough to cause fibrosis in the days and weeks after the procedure.

### Example 10: Bronchial Valve and Application of Electroporation Damage to Tissue

In a tenth example of the first aspect of the invention, the method deploys a one-way bronchial valve as described in Example 8.

Subsequently, an electroporation probe may be delivered as discussed with reference to FIG. 3B. Once deployed in the target area, the electroporation probe applies an electrical field which increases the permeability of cells in the affected area. In some embodiments, this may cause inflammation of the target area which may, in turn, cause fibrosis.

### Example 11: Bronchial Valve and Cryoprobe Freezing

In an eleventh example of the first aspect of the invention, the method deploys a one-way bronchial valve as described in Example 8.

Subsequently, a cryoprobe is delivered as discussed with reference to FIG. 3B, Once deployed in the target area, the cryogenic ablation probe freezes the target area sufficient to cause inflammation of the target area enough to cause fibrosis in the days and weeks after the procedure.

### Example 12: Bronchial Valve and Application of Cryogenic Fluid Freezing

In a twelfth example of the first aspect of the invention, the method deploys a one-way bronchial valve as described in Example 8.

Subsequently, an applicator for a cryogenic fluid is deployed, as described with reference to FIG. 3B. Once deployed in the target area, the cryogenic fluid applicator releases cryogenic fluid, such as liquid nitrogen, liquid argon or liquid helium, which causes rapid freezing upon contact with lung tissue process freezes the target area sufficient to cause inflammation of the target area enough to cause fibrosis in the days and weeks after the procedure.

### Example 13: Bronchial Valve and Application of Thermal Energy by Vapor

In a thirteenth example of the first aspect of the invention, the method deploys a one-way bronchial valve as described in Example 8.

Subsequently, a vapor application device is delivered, as discussed with reference to FIG. 3B. Once deployed in the target area, the device releases vapor which generates heat in the target lung tissue sufficient to cause inflammation of the target area enough to cause fibrosis in the days and weeks after the procedure.

### Examples 14a-14m: Air Removal, Application Inflammatory Response

In examples 14a-14m the method deploys a first suction device delivered as described with reference to FIG. 3A. The suction device causes air to flow out of the target diseased area, causing the local damaged alveoli to collapse, thus resulting in decreased lung volume.

This first step is followed by any one of the subsequent steps outlined in Examples 1 to 13 which cause tissue inflammation.

### Examples 15a-15m: Air Removal, Additional Compaction Step and Application Inflammatory Response

In examples 15a-15m the method deploys a first suction device delivered as described with reference to FIG. 3A. The suction device causes air to flow out of the target diseased area, causing the local damaged alveoli to collapse, thus resulting in decreased lung volume.

Subsequently or post this step, any one of the first of the two step procedures of Examples 1 to 13 is deployed followed on tissue compaction by the associated subsequent step of the example.

1. A method of reducing a volume of functionally impaired lung tissue in a patient in need of treatment for reduced lung function comprising:
applying a first lung volume reduction action to the functionally impaired lung tissue so as to reduce its volume to less than a pre-treatment volume; and
applying a pro-inflammatory stimulus to the functionally impaired lung tissue having reduced volume, that stimulus being sufficient to induce fibrosis in the functionally impaired lung tissue;
wherein the pro-inflammatory stimulus is separate and additional to that of the first lung volume reduction action.

2. A method as claimed in claim 1, wherein the first lung volume reduction action is achieved by compacting the functionally impaired lung tissue.

3. A method as claimed in claim 1, wherein the first lung volume reduction action comprises an application of one or more of a lung volume reduction coil (LVRC), a one-way bronchial valve, a lung sealant adhesive, active removal of air, vapor ablation, radiofrequency ablation, microwave ablation, electroporation or cryogenic ablation.

4. A method as claimed in claim 1, wherein the first lung volume reduction action includes active removal of air.

5. A method as claimed in claim 4, wherein the active removal of air is achieved using an elongate tubular body defining open proximal and distal ends with a passageway suitable for transporting gas extending between the two, the proximal end being in operational connection with a device for producing a lower pressure than that in the functionally impaired lung tissue, and the distal end being deployed into a bronchus supplying air to the functionally impaired tissue such that the distal end is located adjacent to the that functionally impaired tissue.

6. (canceled)

7. A method as claimed in claim 5, wherein the active removal of air is achieved by deploying the distal end of the elongate tubular body into a lobar bronchus supplying air to the functionally impaired tissue.

8. A method as claimed in claim 5, wherein the active removal of air is achieved by applying a reduced pressure to the proximal end of the elongate tubular body such that air in a target area located in the functionally impaired tissue adjacent the distal end of the elongate tubular body is caused to flow into the elongate tubular body, thereby achieving at least partial collapse of alveoli in at least part of the functionally impaired lung tissue.

9. A method as claimed in claim 1, wherein the pro-inflammatory stimulus is applied using a source of heat, cold, sound or electrical energy.

10. A method a claimed in claim 9, wherein the pro-inflammatory stimulus that is applied is one or more of radiofrequency energy, microwave energy, electroporation, ultrasound energy, vapor and cryogenic fluid.

11-13. (canceled)

14. A method as claimed in claim 9, wherein the pro-inflammatory stimulus is a frictional force that is applied using a tissue-engaging surface of a probe.

15. A method as claimed in claim 1, wherein the first lung volume reduction action is applied before the pro-inflammatory stimulus.

16. A method of reducing a volume of functionally impaired lung tissue in a patient in need of treatment for reduced lung function comprising:
deploying an implant device from a constrained delivery configuration to an unconstrained deployed configuration in an airway of a lung, wherein the implant device in the unconstrained deployed configuration is biased to bend the airway of the lung so as to laterally compress a portion of the lung; and
applying a pro-inflammatory stimulus to the functionally impaired lung tissue having reduced volume, wherein the pro-inflammatory stimulus is sufficient to induce fibrosis in the functionally impaired lung tissue.

17. A method as claimed in claim 16, wherein the implant device is a lung volume reduction coil (LVRC).

18. A method as claimed in claim 16, wherein the implant device is delivered to the airway of the lung via a first channel of a delivery device and wherein the pro-inflammatory stimulus is applied using a pro-inflammatory stimulus device delivered via a second channel of the delivery device.

19. A method as claimed in claim 16, wherein the implant device comprises a coating comprising a sclerosing agent, and wherein the pro-inflammatory stimulus comprises elution of the sclerosing agent from the implant device, wherein the sclerosing agent is configured to damage epithelial tissue of the lung and induce fibrosis.

20. A method as claimed in claim 16, wherein the pro-inflammatory stimulus is applied using a source of heat, cold, sound or electrical energy.

21-27. (canceled)

28. A method as claimed in claim 16, further comprising actively removing air from the lung using an elongate tubular body defining open proximal and distal ends with a passageway suitable for transporting gas extending between the two, the proximal end being in operational connection with a device for producing a lower pressure than that in the functionally impaired lung tissue, and the distal end being deployed into a bronchus supplying air to the functionally impaired tissue such that the distal end is located adjacent to the functionally impaired tissue.

29. (canceled)

30. A method as claimed in claim 28, wherein the active removal of air is achieved by deploying the distal end of the elongate tubular body into a lobar bronchus supplying air to the functionally impaired tissue.

31. A method as claimed in claim 28, wherein the active removal of air is achieved by applying a reduced pressure to the proximal end of the elongate tubular body such that air in a target area located in the functionally impaired tissue adjacent the distal end of the elongate tubular body is caused to flow into the elongate tubular body, thereby achieving at least partial collapse of alveoli in at least part of the functionally impaired lung diseased tissue.

32. A system for reducing a volume of functionally impaired lung tissue in a patient in need of treatment for reduced lung function comprising:
a first device, being a lung volume reduction device adapted for placement adjacent to the functionally impaired lung tissue and operating to reduce the volume of the functionally impaired lung tissue to less than a pretreatment volume; and
a second device, being a device capable of applying a pro-inflammatory stimulus to the functionally impaired lung tissue having reduced volume, that stimulus being sufficient to induce fibrosis in the functionally impaired lung tissue;
the first and second devices being deployable together or sequentially, the first followed by the second, to a position adjacent to the functionally impaired lung tissue via a bronchus supplying air to the functionally impaired tissue.

33-34. (canceled)

35. A system as claimed in claim 32, wherein the first device is selected from a lung volume reduction coil, a one-way bronchial valve, a lung sealant adhesive applicator, a catheter capable of active removal of air and a vapor ablation catheter.

36-39. (canceled)

40. A system as claimed in claim 32, comprising an elongate element capable of being passed down a bronchoscope into a patient's lung via bronchi of the patient, the elongate element having a first lumen adapted for removal of air from alveoli of target diseased lung tissue and a second lumen adapted for delivery of one or more of a radiofrequency ablation probe, a microwave ablation probe, an electroporation probe a cryoprobe and an applicator for cryogenic fluid.

41. (canceled)
